(19) United States
(12) Patent Application Publication
(10) Pub. No.: US 2016/0324741 A1
(43) Pub. Date: Nov. 10, 2016

(54) Oral Compositions Containing Zinc

(71) Applicant: The Procter & Gamble Company, Cincinnati, OH (US)

(72) Inventors: Arif Ali Baig, Mason, OH (US); Kimberly Burkhardt, West Chester, OH (US); Carl Edward Catrenich, Fairfield, OH (US); Iain Allan Hughes, Cincinnati, OH (US); Jeanette Marie Swartz, Loveland, OH (US); Yuli Song, Mason, OH (US); Yingkun Jin, Mason, OH (US); George Endel Deckner, Cincinnati, OH (US); Eva Schneiderman, Mason, OH (US)

(21) Appl. No.: 14/706,025

(22) Filed: May 7, 2015

(51) Int. Cl. A61K 8/27; A61K 8/19; A61K 8/81; A61K 8/21

(52) U.S. Cl. CPC A61K 8/27; A61K 8/21; A61K 8/19; A61K 8/8147

(57) ABSTRACT

Oral care composition for preventing and/or reducing calories, including: at least about 0.005%, of metal ions wherein the composition comprises at least about 0.005% of zinc ions and optionally comprises from about 0.001% to about 3%, of stannous ions; at least about 100 ppm of fluoride ions; from about 0.03% to about 10% of a mineral surface active agent selected from homopolymers of itaconic acid, homopolymers of acrylic acid, copolymers of maleic acid and acrylic acid, copolymers of maleic anhydride or acid with methyl vinyl ether, and mixtures thereof; at least 5% of water; less than 10% fused silica, calcium based abrasives, and mixtures thereof; less than 5% of polyphosphates having n+3 or higher; and wherein the weight ratio of metal ion to the mineral surface active agent is equal to or less than about 0.5.

### CROSS REFERENCE TO RELATED APPLICATIONS

This Application claims the benefit of U.S. Provisional Application No. 61/833,323 filed Jun. 10, 2013; and U.S. Provisional Application No. 61/990,726 filed May 9, 2014.

### FIELD OF THE INVENTION

The present invention relates to improved oral compositions containing metal ions including zinc and optionally stannous in combination with a mineral surface active agent and fluoride ions.

### BACKGROUND OF THE INVENTION

The inclusion of metal ions such as zinc and stannous from sources such as zinc lactate and stannous fluoride, respectively, into oral care compositions has been disclosed previously in the literature as well as used in commercially available oral care products. Inclusion of metal ions is often limited by unpleasant side effects such as astringency, taste, and/or staining. However, the underlying efficacy of such metal ions in treating or preventing disease or other negative conditions in the oral cavity has maintained metal ions as a desirable component.

In PCT Application WO 2012/087288 to Porter, et al, the use of metal salts such as zinc salts and stannous salts to provide metal ions in oral care products is disclosed. Such embodiments are disclosed to provide improved delivery of the metal ions to the soft and/or hard tissue of the oral cavity.

Despite the continual development of improved oral care compositions containing metal ions, a need still exists for an oral composition that can appropriately hit the “sweet spot” of formulating to deliver all of the following four benefits: antibacterial efficacy; fluoride uptake; demineralization; reduced stain. The difficulty in balancing all of these competing benefits is well known and the formulator must therefore choose one to three of the benefits for a given composition, and accept that one or more of these benefits will not be provided.

### SUMMARY OF THE INVENTION

It has now been surprisingly discovered that by appropriately balancing the ratio of total metal ions to a selected group of mineral surface active agents, that fluoride uptake may be improved and thereby all four of the benefits necessary to hit the “sweet spot” of oral care efficacy can be realized with one composition. The compositions of the present invention therefore will provide measured antibacterial benefit (by iPGRM) of greater than 50% of a control Crest gum care product; a fluoride uptake (tested as enamel uptake) of better than 20-30 microgram/cm2; a demineralization benefit (as measured by a reduction in enamel solubility of greater than 10% over no treatment); and a reduction in stain of more than 50% versus Crest gum care products.

The improved metal ion containing compositions provide these benefits through the combined effects of a selected ratio of total metal ions (zinc, optionally stannous) to mineral surface active agent selected from homopolymers of itaconic acid, homopolymers of acrylic acid, copolymers of maleic acid and acrylic acid, copolymers of maleic anhydride or acid with methyl vinyl ether, and mixtures thereof; at least 5% water, limiting the fused silica or calcium based abrasive to less than 5% of the composition, and limiting the use of polyphosphates having n+3 or higher in the composition to less than 5%.

The present invention also relates to methods of enhancing therapeutic efficacy while decreasing staining and improving the aesthetic desirability of oral compositions containing zinc with optionally stannous salts, such as stannous fluoride.

These and other features, aspects, and advantages of the present invention will become evident to those skilled in the art from the detailed description which follows.

### DETAILED DESCRIPTION OF THE INVENTION

While the specification concludes with claims, which particularly point out and distinctly claim the invention, it is believed the present invention will be better understood from the following description.

All percentages used herein are by weight of the dentifrice composition, unless otherwise specified. The ratios used herein are molar ratios of the overall composition, unless otherwise specified. All measurements are made at 25° C., unless otherwise specified.

Herein, “comprising” means that other steps and other ingredients which do not affect the end result can be added. This term encompasses the terms “consisting of” and “consisting essentially of”.

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “polymer” as used herein shall include materials whether made by polymerization of one type of monomer or made by two (i.e., copolymers) or more types of monomers. The term “water soluble” as used herein means that the material is soluble in water in the present composition. In general, the material should be soluble at 25° C. at a concentration of 0.1% by weight of the water solvent, preferably at 1%, more preferably at 5%, more preferably at 15%.

The term “phase” as used herein means a mechanically separate, homogeneous part of a heterogeneous system.

The term “majority” as used herein means the greater number or part, a number more than half the total. The term “median” as used herein means the middle value in a distribution, above and below which lie an equal number of values.

The oral composition may be a single phase oral composition or may be a combination of the two or more oral compositions, each in a separate phase. By “single or separate phase” herein is meant that all components of each composition are mixed together in one mixture which may contain liquid, solid and gaseous components. Thus each phase may be homogeneous or non-homogeneous.

If a dual phase oral composition is desired, each oral composition will be contained in a physically separated compartment of a dispenser and dispensed side-by-side. The term “dispenser”, as used herein, means any pump, tube, or container suitable for dispensing toothpaste.

Herein, the terms “tartar” and “calculus” are used interchangeably and refer to mineralized dental plaque biofilms.

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

### Oral Care Composition

The term “oral composition” as used herein means a product that in the ordinary course of usage is retained in the oral cavity for a time sufficient to contact some or all of the dental surfaces and/or oral tissues for purposes of oral activity. In one embodiment, the composition is an “oral care composition” meaning that the composition provides a benefit when used in the oral cavity. The oral composition of the present invention may be in the form of a toothpaste, dentifrice, tooth powder, topical oral gel, mouthrinse, denture product, mouthspray, lozenge, oral tablet, foam, tooth gel, prophy paste, mousse or chewing gum. In one embodiment, the oral composition is in the form of a paste or gel. In another embodiment, the oral composition is in the form of a dentifrice. The oral composition may also be incorporated onto strips or films for direct application or attachment to oral surfaces, or incorporated into floss. The present oral care compositions may be formulated as single phase or dual phase compositions.

The term “dentifrice” as used herein means paste, gel, powder, tablets, or liquid formulations, unless otherwise specified, that are used to clean the surfaces of the oral cavity. The term “teeth” as used herein refers to natural teeth as well as artificial teeth or dental prosthesis.

### Metal Ions

The compositions herein include at least about 0.005%, by weight of the composition, of metal ions; alternatively from about 0.01% to about 2%, by weight of the composition, of metal ions; alternatively from about 0.01% to about 2%, by weight of the composition, of metal ions, including zinc ions and optionally including stannous ions.

### Zinc Ions

The metal ions included herein include at least about 0.005%, by weight of the composition, of zinc ions. In one embodiment, the composition includes from about 0.005% to about 1%, by weight of the composition, of zinc ions, alternatively from about 0.005% to about 1%, by weight of the composition, of zinc ions. The source of such zinc ions may be any zinc salt, including for example, zinc salts selected from zinc citrate, zinc sulfate, zinc glycinate, sodium zinc citrate, zinc lactate, and mixtures thereof. In one embodiment, the source of zinc ions is a zinc salt selected from zinc citrate, zinc lactate, and mixtures thereof. In a preferred embodiment, the zinc ion source is zinc lactate.

The term “zinc ion(s)” as used herein, is defined to mean the zinc that is in a dentifrice or other oral product, and supplied by a source such as zinc salts including zinc lactate. It may refer to the zinc ions that are provided by a zinc source other than zinc salts, added for stabilization purposes.

### Stannous Ions

The metal ions included herein optionally include at least about 0.0001% to about 3%, by weight of the composition, of stannous ions. In one embodiment, the metal ions herein include from about 0.001% to about 0.3%, alternatively from about 0.01% to about 1.5%, by weight of the composition, of stannous ions.

The stannous ion source used herein may include any safe and effective stannous salt and may be selected from stannous fluoride, stannous chloride, stannous pyrophosphate, stannous chloride dihydrate, stannous acetate, stannous gluconate, stannous oxalate, stannous sulfate, stannous lactate, stannous tartrate, and mixtures thereof. In one embodiment, the stannous ion source is selected from stannous fluoride, stannous chloride, and mixtures thereof. In one embodiment, the metal ions include zinc and stannous ions wherein the stannous ion source is stannous fluoride and the zinc ion source is zinc lactate.

The term “stannous ion(s)” as used herein, is defined to mean the stannous that is in a dentifrice or other oral product, and supplied by a source such as stannous salts including stannous fluoride. It may refer to the stannous ions that are provided by a stannous salt other than stannous fluoride, added for stabilization purposes.

The combined stannous salts will be present in an amount of from about 0.05% to about 11%, by weight of the total composition. Preferably, the stannous salts are present in an amount of from about 0.1 to about 7%, more preferably from about 0.4% to about 3%. Formulations typically include stannous levels, provided by stannous fluoride and other stannous salts, ranging from about 3,000 ppm to about 15,000 ppm stannous ions in the total composition.

Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al.

### Fluoride

The compositions herein include at least about 100 ppm, by weight of the composition, of fluoride ions. In one embodiment, the compositions herein include from about 100 ppm to about 15,000 ppm, by weight of the composition, of fluoride ions. The term “fluoride ion(s)” as used herein, is defined to mean the fluoride that is in a dentifrice or other oral product, and supplied by a source such as metal salts including stannous fluoride. It may refer to the fluoride ions that are provided by a fluoride source other than stannous fluoride.

Fluoride ion sources include sodium fluoride, stannous fluoride, indium fluoride, amine fluoride and sodium monofluorophosphate. Stannous fluoride is a preferred soluble fluoride source. This ingredient may serve as both a/the stannous source and fluoride source. Norris et al., U.S. Pat. No. 2,946,725, issued Jul. 26, 1960, and Widder et al., U.S. Pat. No. 3,678,154 issued Jul. 18, 1972, disclose such fluoride sources as well as others.

The present compositions may contain a soluble fluoride ion source capable of providing from about 50 ppm to about 3500 ppm, and preferably from about 500 ppm to about 3000 ppm of free fluoride ions. To deliver the desired amount of fluoride ions, fluoride ion sources may be present in the total oral composition at an amount of from about 0.1% to about 5%, preferably from about 0.2% to about 1%, and more preferably from about 0.3% to about 0.6%, by weight of the total composition delivered to the oral cavity.

### Mineral Surface Active Agent

The compositions herein include at least about 0.01%, by weight of the composition, of a mineral surface active agent (MSA). In one embodiment, the compositions include from about 0.01% to about 35%, alternatively from about 0.03 to about 20%, alternatively from about 0.03% to about 10%, by weight of the composition, of the mineral surface active agent.

Mineral surface active agents useful herein include those selected from homopolymers of itaconic acid, homopolymers of acrylic acid, copolymers of maleic acid and acrylic acid, copolymers of maleic anhydride or acid with methyl vinyl ether, and mixtures thereof. In one embodiment, the mineral surface active agent is selected from homopolymers of acrylic acid. In one embodiment, the mineral surface active agent is selected from copolymers of maleic anhydride or acid with methyl vinyl ether. In one embodiment, the mineral surface active agent is selected from copolymers of maleic acid and acrylic acid. In one embodiment, the mineral surface active agent is selected from homopolymers of itaconic acid.

The “mineral” descriptor is intended to convey that the surface activity or substantivity of the surface-active agent is toward mineral surfaces such as calcium phosphate minerals or teeth. These agents show affinity for binding metal ions, in particular by stannous ion chelation, as evidenced by ionic fluoride release from stannous fluoride (SnF₂) and provision of increased ionic form of fluoride upon binding of the stannous. Effective agents also show surface reactivity toward calcium phosphate minerals, and are thus expected to retard calculus or tartar formation. The agents may also provide stain control, surface conditioning and antierosion benefits. Ideally, these agents will bind the stannous but will still enable the combined mixture to provide the desired tartar control, stain control, and surface conditioning, without having a negative effect on the efficacy of stannous fluoride for the control of dental caries, oral malodor and periodontal diseases including gingivitis.

Mineral surface agents useful herein include the synthetic anionic polymers including polyacrylates and copolymers of maleic anhydride or acid and methyl vinyl ether (e.g., GANTREZ), as described, for example, in U.S. Pat. No. 4,627,977, to Gaffar et al. Polymeric polycarboxylates disclosed in U.S. Pat. No. 4,138,477, Feb. 6, 1979 and U.S. Pat. No. 4,183,914, Jan. 15, 1980 to Gaffar et al. and include copolymers of maleic anhydride or acid with another polymerizable ethylenically unsaturated monomer, such as methyl vinyl ether (methoxyethylene). Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride with methyl vinyl ether having a molecular weight (M.W.) of about 30,000 to about 1,000,000. These copolymers are available for example as GANTREZ AN 139 (M.W. 500,000), AN 119 (M.W. 250,000) and S-97 Pharmaceutical Grade (M.W. 70,000), of GAF Chemicals Corporation.

Other MSAs useful herein include the ACUSOL line of homopolymers of acrylic acid and copolymers of maleic acid and acrylic acid commercially available from Dow Chemicals.

Other MSAs useful herein include polymers of itaconic acid. The itaconic polymers herein are the homopolymers of itaconic acid (methylenesuccinic acid), having a molecular weight of from about 1000 to about 20,000. Such polymers are commercially available from Itaconix, USA, for example, the linear polyitaconic acid partially neutralized with sodium salt polymers sold under the name ITACONIX DSP2K (molecular weight about 5,000; CAS#26099-89-8), ITACONIX DSP1K (molecular weight about 2,000), ITACONIX DSP5K, and ITACONIX DSP10K.

### Ratio of Metal Ion to Mineral Surface Active Agent

Although combinations of metal ions and mineral surface active agents have been taught for use in oral care compositions in the past, it has now been surprisingly found that the weight ratio of these materials is important to fluoride uptake. Therefore the weight ratio of metal ion to the mineral surface active agent is equal to or less than about 0.5. In one embodiment, the ratio is less than about 0.35. In another embodiment, the ratio is less than about 0.3.

### Orally Acceptable Carrier Materials

In preparing the present compositions, it is desirable to add one or more carrier materials or excipients to the compositions in addition to the materials set forth above.

The term “orally acceptable carrier” as used herein includes safe and effective materials for use in the compositions of the present invention. Such materials are conventional additives in oral care compositions including but not limited to, buffers, abrasives such as silica, alkali metal bicarbonate salts, thickening materials, humectants, water, surfactants, titanium dioxide, flavor system, sweetening agents, xylitol, coloring agents, and mixtures thereof. These carriers may be included at levels typically from about 5% to about 99%, preferably from about 20% to about 98%, and more preferably from about 30% to about 95%, by weight of the oral care composition. Examples of such carriers are described in the following paragraphs.

### Water

The compositions herein include at least 5%, by weight of the composition, of water, alternatively at least about 10%, alternatively at least about 20%, alternatively at least about 25%, at least about 30%, by weight of the composition, of water.

Water employed in the preparation of commercially suitable oral compositions should preferably be of low ion content and free of organic impurities. In the oral composition, water may comprise from 0% up to about 95%, and preferably from about 5% to about 50%, by weight of the composition herein. The amounts of water include the free water which is added plus that which is introduced with other materials, such as with sorbitol, silica, surfactant solutions, and/or color solutions.

### Abrasive

Compositions of the present invention may include an abrasive. Abrasives useful herein include any abrasives that have stability with stannous. In one embodiment, the abrasive is selected from precipitated silica, polymethylsilsesquioxane silicone resin particles, and mixtures thereof.

Abrasives such as fused silica and calcium-based abrasives (such as calcium, pyrophosphate, calcium carbonate, calcium phosphate) have been found to be less preferred, therefore the compositions herein include less than 10%, by weight of the composition of fused silica, calcium based abrasives, and mixtures thereof, alternatively less than 8%, alternatively less than 5%, alternatively less than 1%, alternatively are substantially free of such materials. As used herein, “substantially free of” means that no material is intentionally added to the composition, but may be present in very small, not readily measurable amounts, such as where the materials are present as an impurity in another material added to the composition. In one embodiment, the composition is free of fused silica, calcium pyrophosphate, tricalcium phosphate, calcium polymetaphosphate, beta calcium pyrophosphate, and calcium carbonate.

The abrasives useful herein generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, “Zeodent”, particularly the silica carrying the designation “Zeodent 119”. The types of silica dental abrasives useful in the toothpastes of the present invention are described in more detail in Wason, U.S. Pat. No. 4,340,583, issued Jul. 29, 1982. Other suitable silica abrasives are described in Rice, U.S. Pat. Nos. 5,589,160; 5,603,920; 5,651,958; 5,658,553; 5,716,601 and in White, Jr., et al. U.S. Pat. No. 6,740,311. The abrasive in the oral composition compositions described herein is generally present at a level of from about 6% to about 70% by weight of the composition. Preferably, oral compositions contain from about 10% to about 50% of abrasive, by weight of the oral composition.

### Buffering Agent

The present compositions may contain a buffering agent. Buffering agents, as used herein, refer to agents that can be used to adjust the pH of the compositions to a range of about pH 3.0 to about pH 10. The oral composition will typically have a pH of from about 4 to about 7, preferably from about 4.5 to about 6.5, and more preferably from about 5 to about 6.

Suitable buffering agents include alkali metal hydroxides, carbonates, sesquicarbonates, borates, silicates, phosphates, imidazole, and mixtures thereof. Specific buffering agents include monosodium phosphate, trisodium phosphate, sodium benzoate, benzoic acid, sodium hydroxide, potassium hydroxide, alkali metal carbonate salts, sodium carbonate, imidazole, pyrophosphate salts, citric acid, and sodium citrate. Preferred buffers would be those that control the pH in the target range without complexing stannous ions. Preferred buffering agents include acetic acid, sodium acetate, citric acid, sodium citrate, benzoic acid and sodium benzoate. Buffering agents are used at a level of from about 0.1% to about 30%, preferably from about 1% to about 10%, and more preferably from about 1.5% to about 3%, by weight of the present composition.

### Additional Carriers

Thickening agents may be used herein, such as those selected from carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can be used in an amount from about 0.1% to about 15%, by weight of the oral composition.

The compositions herein may include from about 0% to 70%, and preferably from about 15% to 55%, by weight of the oral composition, of a humectant. Suitable humectants for use in the invention include glycerin, sorbitol, polyethylene glycol, propylene glycol, xylitol, and other edible polyhydric alcohols.

The compositions herein may also include surfactants, also commonly referred to as sudsing agents. Mixtures of surfactants can be used. Suitable surfactants include anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Many suitable anionic surfactants are disclosed by Agricola et al., U.S. Pat. No. 3,959,458, issued May 25, 1976. Nonionic surfactants which can be used in the compositions of the present invention can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene, polyoxyethylene sorbitan esters (sold under trade name Tweens), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The nonionic surfactant poloxamer 407 is one of the most preferred surfactant because the poloxamer has been discovered to help reduce the astringency of the stannous. The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Many of the suitable nonionic and amphoteric surfactants are disclosed by Gieske et al., U.S. Pat. No. 4,051,234, issued Sep. 27, 1977. The present composition typically comprises one or more surfactants each at a level of from about 0.25% to about 12%, preferably from about 0.5% to about 8%, and most preferably from about 1% to about 6%, by weight of the composition.

The compositions herein may include from about 0.25% to about 5%, by weight of the composition of titanium dioxide; may contain from about 0.01%, to about 5%, by weight of the composition, of a coloring agent such as one in a 1% aqueous solution.

The compositions herein may include a flavor component. Suitable flavoring components include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) and mixtures thereof. A flavor system is generally used in the compositions at levels of from about 0.001% to about 5%, by weight of the composition.

Sweetening agents can be added to the compositions. These include saccharin, dextrose, sucrose, lactose, xylitol, maltose, levulose, aspartame, sodium cyclamate, D-tryptophan, dihydrochalcones, acesulfame, and mixtures thereof. Sweetening agents and generally used in toothpastes at levels of from about 0.005% to about 5%, by weight of the composition.

The present invention may also include other agents to provide antimicrobial benefits. These agents may be included at levels which do not prevent the interaction between stannous and the MSA. Included among such antimicrobial agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, among others. Triclosan monophosphate is an additional water soluble antimicrobial agent. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are examplary of typical quaternary ammonium antibacterial agents. Other compounds are bis[4-(R-amino)-1-pyridinium] alkanes as disclosed in U.S. Pat. No. 4,206,215, issued Jun. 3, 1980, to Bailey. Other antimicrobials such as copper bisglycinate, copper glycinate, zinc citrate, and zinc lactate may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, to Gieske et al. Specific antimicrobial agents include chlorhexidine, triclosan, triclosan monophosphate, and flavor oils such as thymol. Triclosan and other agents of this type are disclosed in U.S. Pat. No. 5,015,466, issued to Parran, Jr. et al. and U.S. Pat. No. 4,894,220, to Nabi et al. The water insoluble antimicrobial agents, water soluble agents, and enzymes may be present in either the first or second oral compositions if there are two phases. These agents may be present at levels of from about 0.01% to about 1.5%, by weight of the oral composition.

### Polyphosphates

Polyphosphates may be included in the compositions herein, but linear polyphosphates having n+3 or higher should be limited due to their ability to undergo hydrolysis in formulations containing water, such as the aqueous formulations herein. Therefore, the compositions herein include less than 5%, by weight of the composition, of linear polyphosphates having n+3 or higher; and are preferably substantially free, alternatively are free of such materials. A polyphosphate is generally understood to consist of two or more phosphate molecules arranged primarily in a linear configuration, although some cyclic derivatives may be present. The longer-chain polyphosphate salts include tetrapolyphosphate and hexametaphosphate, among others. Polyphosphates larger than tetrapolyphosphate usually occur as amorphous glassy materials. Examples of such polyphosphates are the linear “glassy” polyphosphates having the formula:

XO(XPO₃)ₙX

wherein X is sodium, potassium or ammonium and n averages from about 6 to about 125. Preferred are polyphosphates manufactured by FMC Corporation which are commercially known as Sodaphos (n≈6), Hexaphos (n≈13), and Glass H (n≈21). It is also known that polyphosphates with an average chain length greater than about 4 will react with ionic fluoride in oral compositions at ambient temperature and produce monofluorophosphate ions, in addition to altering the pH of the composition. This reaction compromises the efficacy of the oral composition and its ability to provide stable ionic fluoride and polyphosphate to the oral surfaces.

### Botanicals

The oral care compositions herein may further comprises at least one botanical or extract thereof selected from chamomile, cinnamon, citrus, clove, echninacea, eucalyptus, fennel, ginger, green tea, hop, magnolia, nutmeg, peppermint, pomegranate, rosemary, saffron, sage, spearmint, star anise, turmeric, wintergreen, extracts thereof and mixtures thereof. A lengthy list of botanicals that may be useful herein include those found in U.S. Pat. No. 7,736,629 B2 to Kamath, et al., Jun. 15, 2010. In one embodiment, the botanical or extract thereof is selected from Hops, extracts thereof and mixtures thereof. One example of a botanical useful herein is the commercially available CLEAN BETA BIO HOPS material from Hopsteiner.

Hops are the female seed cones of a hop species, Humulus lupulus. Hops are used extensively in brewing for many benefits, including an antibacterial effect that favors the activity of brewer's yeast over less desirable microorganisms. Hops can be subjected to CO₂ and ethanol extraction procedures, after which the major components are alpha acids (50-70%), beta acids (20-35%), hop oils (3-7%) and resins (5-15%).

In one embodiment, the oral care compositions herein have improved efficacy for preventing and/or reducing caries and include from about from about 0.05% to about 1%, by weight of the composition, of metal ions wherein the composition comprises from about 0.05% to about 1%, by weight of the composition, of zinc ions and optionally comprises from about 0.01% to about 1.5%, by weight of the composition, of stannous ions; from about 100 ppm to about 15,000 ppm, by weight of the composition, of fluoride ions; from about 0.03% to about 10%, by weight of the composition, of a mineral surface active agent selected from homopolymers of itaconic acid, homopolymers of acrylic acid, copolymers of maleic acid and acrylic acid, copolymers of maleic anhydride or acid with methyl vinyl ether, and mixtures thereof; at least 5%, by weight of the composition, of water; less than 5%, by weight of the composition, of fused silica, calcium based abrasives, and mixtures thereof; less than 5%, by weight of the composition, of polyphosphates having n+3 or higher; and wherein the weight ratio of metal ion to the mineral surface active agent is equal to or less than about 0.5.

### Dual Compartment/Two-Step

The compositions herein may be used alone, or in combination with other compositions in a two or more step regimen. In one embodiment, the compositions herein are packaged in a tube or other dispensing package and then combined into a secondary package with another product such as a hydrogen peroxide-containing gel. Methods of using a first composition according to the oral care compositions herein for brushing teeth and then using a second composition containing a source of peroxide are also desirable. A second composition including a peroxide source may include, for example, a peroxide selected from hydrogen peroxide, calcium peroxide, urea peroxide, and mixtures thereof. Such a secondary composition may contain from about 0.01% to about 10%, alternatively from about 0.1% to about 5%, alternatively from about 0.2% to about 3%, alternatively from about 0.3% to about 0.8% of a peroxide source, by weight of the oral composition.

### Methods

The present invention also relates to methods of preventing caries wherein the method includes the steps of: applying the oral care compositions herein to the teeth; rinsing the composition from the oral cavity; and expectorating, wherein the method is conducted on an at least daily basis for a period of at least one week.

### Efficacy Measures

### Overall Performance

Overall performance of the present compositions may be defined in terms of an efficacy score/stain score ratio, wherein efficacy is measured using the in vitro Plaque Glycolysis and Regrowth Model (i-PGRM), and stain is measured using the in vitro Pellicle Tea Stain Model (i-PTSM). The present compositions provide an efficacy score to stain score ratio of at least 1.2, which represents a realistic improvement in that sufficient therapeutic efficacy is maintained while achieving a reduction in staining Improvement in formulation astringency is defined as greater than 50% increase in formulation mouth feel parameters such as dry mouth, and clean mouth indices as defined in controlled consumer testing. Effectiveness for control of supragingival calculus is defined by activity in prevention of plaque calcification using the Modified Plaque Growth and Mineralization assay.

Antimicrobial Activity

The metal ion concentration and bioavailability required for the provision of therapeutic actions may differ for different clinical actions, for example, caries vs. gingivitis. However, it is critical to establish a minimum antimicrobial activity level, since the therapeutic activity of metal ions can be compromised below this level. It is especially important to maintain efficacy in compositions wherein binding of the metal ions occurs, since metal binding can easily lead to loss of antimicrobial activity. Herein, the minimum efficacy provided by the metal ion source is defined in terms of effects in producing metabolic inhibition of dental plaque bacterial biofilms, which are responsible for numerous undesirable intraoral conditions. Efficacy is thus defined in terms of a noticeable and significant reduction in in situ plaque metabolism as measured using the in vitro Plaque Glycolysis and Regrowth Model (i-PGRM), developed in our laboratories. The efficacy of stannous containing compositions for gingivitis can be directly compared to a stannous-containing dentifrice formulation such as described in U.S. Pat. No. 5,004,597 to Majeti, et al. or to a marketed dentifrice containing stannous fluoride, Crest Gum Care.

The i-PGRM is a technique where plaque is grown from human saliva, and treated with agents designed to produce various levels of antimicrobial activity. The purpose of this technique is to provide a simple and quick method for determining if compounds have a direct effect on the metabolic pathways that plaque microorganisms utilize for the production of toxins which adversely affect gingival health. In particular, the model focuses on the production of organic acids including lactic, acetic, propionic, and butyric. This method utilizes plaque grown on polished glass rods which have been dipped in saliva overnight, soy broth and sucrose for 6 hours, and saliva again overnight. The plaque mass grown on the glass rods is then treated for 1 minute with a 3:1 water to dentifrice slurry. The mass is then placed in a soy broth/sucrose solution for 6 hours and the pH of the incubation solution is measured at the end of the 6 hours. Thus, there are measures of pre-incubation pH and post incubation pH for both test formulations and controls. This testing is typically done with a number of replicates to minimize experimental variances, and a mean pH is calculated from the replicates. Due to strong reactivity with saccharolytic organisms, compositions containing high levels of bioavailable stannous produce significant inhibition of plaque acid generation in the i-PGRM assay. This enables formulation variations to be compared for stability and bioavailability of stannous with relative ease.

Metal ions are found in the oral compositions described herein in an effective amount to provide a desired i-PGRM score. The desired i-PGRM score is measured relative to non-stannous containing formulations (negative control) and to stannous-containing formulations (positive control) such as described in U.S. Pat. No. 5,004,597 to Majeti et al. Most preferable i-PGRM scores are significantly different from placebo controls and ideally similar to those provided by conventional stannous fluoride compositions proven effective for reducing plaque and gingivitis. Research has demonstrated that effective gingivitis efficacy can be anticipated for compositions providing at least about 60%, preferably at least about 70%, and more preferably at least about 80% of an effective stannous-containing dentifrice such as shown below in the Examples.

The i-PGRM score is calculated according to the formula:

$$i \text{-} P G R M Score = 100 \% \times \frac{( Test product mean pH - Non \text{-} Stannous Control mean pH )}{( Stannous Control mean pH - Non \text{-} Stannous Control mean pH )}$$

The mean pH values refer to incubation media pH's obtained following treatment and sucrose challenge. The non-stannous control plaque samples produce large amounts of acid, and hence their pH's are lower than that of plaque samples treated with the positive control. The effectiveness of a formulation prepared from the combination of a metal ion source and MSA will ideally be comparable to the stannous-containing control, and hence ideal i-PGRM score should approach 100%.

### Staining Reduction

Tooth staining is a common undesirable side effect of the use of stannous fluoride compositions. Improved stannous fluoride dentifrices described herein provide reduced dental stain formation resulting from more efficient stannous delivery from stannous bound to the MSA. The staining of the tooth surface typically caused by stannous is measured in the clinical situation by using a stain index such as the Lobene or Meckel indices described in the literature. An in vitro staining model has also been developed which provides quantitative estimates for stannous fluoride formulation staining potential which correlate well with clinical observations. Formulations can thus be tested in advance of clinical examination using these methods.

The in-vitro Pellicle Tea Stain Model (i-PTSM) is a technique where an in vitro plaque biomass is grown on glass rods from pooled human stimulated saliva over the course of three days. The plaque biomass is treated with 3:1 water to dentifrice supernatants, where abrasive and insoluble solids have been removed via centrifugation, to determine potential dental staining levels of the various agents. The purpose of this technique is to provide a simple and quick method for determining if compounds have a direct effect on the amount of dental plaque stain. This method utilizes plaque grown on polished glass rods from pooled human saliva with treatments of 5 minutes each, followed by a 10 minute tea treatment. The treatment regimen is repeated at lest three times before the plaque mass is digested off the rods, filtered and absorbance at 380 nm is measured. This testing is typically done with a number of replicates to minimize experimental variances, and a mean absorbance is calculated from the replicates.

It has been found that the stain, which is typically produced by effective stannous fluoride is reduced by combining the stannous fluoride with one or a mixture of the MSA's discussed above. The benefit of reducing the staining caused by stannous is achieved with the present compositions without significantly compromising the efficacy of the stannous, fluoride, and MSA. The amount of staining resulting from the oral compositions of the present invention is significantly lower than the amount of staining resulting from typical dentifrices containing stannous. The term “reduced” as used herein means a statistically significant reduction. Therefore, reducing the staining of stannous means that the amount of stain is statistically significantly reduced compared to a stannous-containing positive control.

Not reducing the efficacy of the stannous means the efficacy of the stannous is not statistically significantly reduced relative to a stannous-containing positive control. Alternatively, stain may be measured relative to typical oral compositions, which do not contain stannous fluoride or another antimicrobial agent which is known to stain. Therefore, the compositions may be measured relative to very little to no stain.

The i-PTSM score can be calculated from this staining assay according to the formula:

$$i \text{-} P T S M Score = 100 \% \times \frac{Test Product Mean Absorbance}{( Stannous Control Mean Absorbance )}$$

The mean absorbance values refer to digested plaque colorimetric values obtained following dentifrice treatments and tea rinsing challenge. The stannous control used is typically a high staining stannous fluoride formulation. The stannous control samples produce large amounts of tea absorption and hence increased colorimetric absorbance. Thus, the i-PTSM score is a measure of the relative level of staining. The lower the score, the lower the level of staining. The combination of a stannous ion source and MSA provides a reduction in staining and will ideally have a i-PTSM score of less than about 75%, preferably less than 60%, more preferably less than 50%, most preferably less than 25%.

Ratio of i-PGRM Score to i-PTSM Score

A key descriptor of the improvement in stannous compositions provided herein is the ratio of efficacy of stannous in comparison to staining potential, these being key consumer concerns. The effectiveness of the oral composition of the present invention will be measured by a ratio of i-PGRM score to i-PTSM score.

The ratio of i-PGRM score to i-PTSM score is calculated according to the formula:

Ratio=i-PGRM score/i-PTSM score

In accordance with the present invention, the ratio developed using these methods should be at least about 1.2 for significant improvements in stannous formulation efficacy relative to tooth staining side effects. The ratio is preferably above about 1.3, more preferably above about 1.5, and most preferably above about 2.0. If there is little to no stain occurring, the ratio approaches infinity, which is preferred.

### Binding of Stannous

As discussed above, effective stabilization of stannous (efficacy with reduced side effects) may be accomplished by in situ binding or complexation of stannous ion with the mineral surface active agent (MSA). In mixed compositions containing stannous fluoride, evidence of binding of stannous is readily observed by potentiometric detection of available ionic fluoride. For example, binding of stannous with polyphosphate MSA ligand results in exchange of fluoride from stannous fluoride and release as ionic fluoride into solution. Relevant measures of stannous binding can be assessed by this technique because fluoride is the strongest ligand in the system after the MSA binding agent. Thus, fluoride release is illustrative of stannous binding by the MSA under these conditions.

### Astringency Reduction

Astringency is an additional side effect of many stannous containing compositions which is significantly improved in the present compositions comprising the MSA's in combination with metal ions. The astringency of formulations can be measured in intraoral panels, where subjects assess mouth condition before and after tooth brushing with the test formulations. In these studies, time dependent studies can be made of dentifrice effects on consumer subjective responses. In one protocol, panelists began a conditioning series by having teeth cleaned with vigorous self oral hygiene including brushing for two three minute periods, flossing and disclosing to ensure complete plaque removal. Subjects are then assigned their test product and instructed to brush with twice per day as usual. For these tests, subjects reported in the morning to a clinic prior to any oral hygiene or food or beverage consumption. Panelists are then asked to fill out a subjective mouth feel assessment questionnaire including questions on tooth clean feeling, smooth teeth feeling and clean mouth feeling as well as assessments of mouth moisture. Panelists then brushed for one minute with assigned oral product. At this point, before lunch and before dinner (late p.m.) subjects again filled out subjective mouth feel questionnaire. Acceptability of the present compositions was found to be comparable to conventional sodium fluoride (NaF) and tartar control dentifrices respectively.

The present invention also relates to a method of treating gingivitis and plaque with reduced staining, by using the present compositions. Additionally provided are methods of providing oral care compositions, which have caries, gingivitis, plaque, tartar, stain, sensitivity, aesthetics, breath, mouthfeel, and cleaning benefits. The benefits of these compositions may increase over time when the composition is repeatedly used. Specifically, the method of treatment will include reducing the gingivitis and plaque, as measured by the i-PGRM, while reducing the staining caused by oral composition containing stannous, as measured by the i-PTSM. The ratio of the i-PGRM score to i-PTSM stain model score is above about 1.2.

The present invention also relates to methods for reducing the incidence of calculus on dental enamel and to methods for providing desirable mouth aesthetic benefits including reduced astringency and oral surface conditioning effects. The benefits of these compositions may increase over time when the composition is repeatedly used.

Methods of treatment include preparing an oral composition as set forth herein and administering the composition to the subject. Administering to the subject may be defined as having the oral care composition contact the tooth surfaces of the subject by brushing with a dentifrice or rinsing with a dentifrice slurry. Administration may also be by contacting the topical oral gel, mouthrinse, denture product, mouthspray, oral tablet, lozenge, or chewing gum with the tooth surfaces. The subject may be any person or animal in need of treatment or prevention of oral conditions including plaque, gingivitis, tartar, stain, and sensitivity. By “animal” is meant to include in particular household pets or other domestic animals, or animals kept in captivity.

### EXAMPLES

The following examples and descriptions further clarify embodiments within the scope of the present invention. These examples are given solely for the purpose of illustration and are not to be construed as limitations of the present invention as many variations thereof are possible without departing from the spirit and scope.

The following are the test methods used in the Examples herein. These are common in-vitro test methods frequently utilized with oral care compositions to predict in-vivo results.

In-Vitro Pellicle Tea Stain Model (iPTSM)

Tooth staining is a common undesirable side effect of the use of stannous fluoride compositions. Improved stannous fluoride dentifrices described herein provide reduced dental stain formation resulting from more efficient stannous delivery from stannous bound to the polymeric mineral surface active agent. The staining of the tooth surface typically caused by stannous is measured in the clinical situation by using a stain index such as the Lobene or Meckel indices described in the literature. For rapid screening of technologies to help mitigate stannous induced staining, an in vitro lab method is used that provides quantitative estimates of stain prevention potential of stannous fluoride formulations. This method, called iPTSM (in-vitro pellicle stain model), has been shown to correlates well with clinical observations.

The in vitro pellicle tea stain model (iPTSM) is a technique where an in vitro plaque biomass is grown on glass rods from pooled human stimulated saliva over the course of three days. The plaque biomass is treated with agents to determine potential dental staining levels of the various agents. The purpose of this technique is to provide a simple and quick method for determining if compounds have a direct effect on the amount of dental plaque stain. This method utilizes plaque grown on polished glass rods from pooled human saliva with treatments of 5 minutes duration, followed by a 10 minute tea treatment. A trial of this in-vitro model can be completed in five days during which up to 12 treatments, including controls can be evaluated.

### 1. Roughening Glass Rods

Polish new glass rods (5 mm×90 mm) approximately 25 mm from the untapered end on a lathe with silicon carbide paper of 240, 320, 400, and 600 grit used sequentially. After the initial polishing, polish the rods with 600 grit paper only before each test.

### 2. Saliva Collection & Preparation

Collect saliva daily from a panel of 5-10 people by paraffin stimulation and refrigerate at 4° C. till needed. Pool saliva carefully (so not to pour in wax/mucus) and mix thoroughly.

### 3. Day 1:

Clean glass rods by sonicating with dilute HCl acid, rinse, dry, and polish with 600 grit silicon carbide paper. Rinse rods again with DI water and dry. Insert rods into holders, adjust depth with the depth gauge on the treatment rack, and secure rods in place with rubber O-rings.

In the early afternoon, pipette 7 ml of saliva, to which 0.1% sucrose has been added, into 16×75 mm test tubes in a dipping rack. Sucrose is added to saliva on the first day only. Place the rod holders in a modified 37° C. incubator designed to dip roughened glass rods into test tubes to a depth of 1.5 cm at 1 rpm. Dip rods overnight. The design of the incubator is fully shown in Attachment 1. Prepare plaque growth media described above and autoclave for Day 2 (saliva is added on Day 2 before use).

### 4. Day 2:

In the morning, add saliva to plaque growth media and mix thoroughly. Pipette 7 ml of plaque growth media into new 16/75 mm test tubes in new dipping rack. Remove old rack of used tubes, place new dipping rack into incubator, and dip rods for six hours MINIMUM before replacing rods into fresh saliva for overnight dipping.

### 5. Day 3

On the morning of the third day, pipette 10 ml of DI water into 17×100 mm test tubes in the second and third rows of the treatment rack. This applies to dentifrice treatments only. Rinse solutions may or may not have water rinse tubes in the treatment rack. Pipette fresh pooled saliva into a dipping rack and set aside. Begin tea preparation by adding 550 ml to a glass beaker and heating it in the microwave for 10 minutes. At the end of ten minutes, carefully remove beaker from microwave and drop in a magnetic stir bar to dissipate the possible presence of a super-heated water core. Place 5 Lipton tea bags and a Celsius thermometer into the water and stir on a hot plate. This solution needs to be monitored to insure that it will be no hotter than 50° C. when tea treatment begins. While tea treatment is heated and mixed, prepare dentifrice slurries (1 part dentifrice to 3 parts water, also called a 1 in 4 dilution) using a handheld homogenizer for 30 seconds. Centrifuge slurries for 15 minutes at 10000 rpm. Rinse or active solutions are treated neat. Pipette 7 ml of 50° C. tea solution into a separate dipping rack. Add 5 ml of supernatant/rinse to 16×75 mm glass test tubes in the first row of the treatment rack. Turn off incubator dipping mechanics and remove old saliva dipping rack. Remove all rod holders from the incubator and place submerged rods into old saliva dipping rack to prevent drying over. Using one rod holder at a time, treats by soaking for 5 minutes in the treatment rack. If applicable, wash rods with 2×10 sec dipping in the test tubes containing the DI water in the treatment rack. Place rod holders into prepared tea solution dipping rack and soak for 10 min. Repeat this process with all four rod holders, returning holders to dipping rack to prevent drying out. Place fresh saliva dipping rack into incubator. Return rods to the incubator after treatment/tea soak and dip in fresh saliva for at MINIMUM of 1 hour. This treatment cycle is repeated two more times with fresh treatment/tea/saliva solutions for a total of 3 treatments in a day. After the last treatment, return rods to the incubator and dip overnight in fresh saliva.

### 6. Day 4:

On the morning of the fourth day, turn off incubator dipping mechanics and remove rods from the saliva. Allow rods to dry are then weigh to the nearest 0.1 mg. Record weight and calculate mean dry plaque biomass weights and standard deviations. Place rods into clean sterile cap-able test tubes containing 3 ml of 0.5M KOH, cap tightly and digest overnight at 37° C.

### 7. Day 5:

On the fifth day, remove rods from the incubator and allow cooling. Vortex glass rods to insure all deposits are homogenized. Remove rods from test tubes, filter the solution through 0.45 μm cellulose acetate syringe filters and an read absorbance values for each rod at 380 nm in spectrophotometer. Record results and use absorbance values to calculate mean absorbance value per treatment, standard deviations per treatment, mean absorbance per mg plaque, Standard deviations of mean absorbance per mg plaque, and % increase in absorbance per mg plaque vs. control according to the following equation,

% Stain Potential=((Test Product Abs/biomass−Non stannous control Abs/Biomass)/(High Stannous control Abs/Biomass−Non stannous control Abs/Biomass))*100

### 8. Controls:

Usual controls for a dentifrice iPTSM run are 1 in 4 supernatants of Crest Cavity Protection (negative control) and Crest Gum Care (positive control). For experiments on mouth rinses or solutions, usual controls include water, placebo mouth rinse formulations (negative controls) and Peridex mouthwash (positive control). There are 4 rods per treatment group.

In-Vitro Plaque Glycolysis and Regrowth Model (iPGRM)

Anytime a metal complexing agent is used to mitigate side effects via stannous binding, there's a risk of compromising bioavailibility and efficacy of stannous ion. Hence, it is critical to establish a minimum antimicrobial activity level that corresponds to therapeutic efficacy and that below such level stannous related performance is significantly affected. We have developed an in-vitro method to measure bioavailibility and antimicrobial efficacy of stannous ion in formulations, where efficacy is defined in terms of noticeable and significant reduction in plaque metabolic activity as measured by reduction in acid formation by bacteria when exposed to sugar.

In-vitro plaque glycolysis and regrowth model (iPGRM) is a technique where plaque is grown on glass rods from human saliva and treated with various agents to determine antiglycolytic activity of treatments. The general purpose of this technique is to provide a simple and quick method for determining if compounds have an influence on the metabolic pathways that plaque microorganisms utilize for the production of toxins that adversely affect gingival health. In particular, the model focuses on the production of organic acids such as lactic, acetic, butyrate, etc. A trial of this in vitro model can be completed in three days during which up to 12 treatments (4 replicates/treatment), including controls can be evaluated. Following are the main steps involved in iPGRM.

### Roughening Glass Rods

Polish new glass rods (5 mm×90 mm) approximately 25 mm from the untapered end on a lathe with silicon carbide paper of 240, 320, 400, and 600 grit used sequentially. After the initial polishing, polish the rods with 600 grit paper after each test.

### Saliva Collection & Preparation

Collect saliva daily from a panel of 5-10 people by paraffin stimulation and refrigerate at 4° C. till needed. Pool saliva carefully (so not to pour in wax/mucus) and mixed thoroughly.

### 3. Day 1:

Clean glass rods by sonicating with dilute HCl acid, rinse, dry, and polish with 600 grit silicon carbide paper. Rinse rods again with DI water and dry. Insert rods into holders, adjust depth with the depth gauge on the treatment rack, and secure rods in place with rubber o-rings.

In the afternoon, pipette 7 ml of saliva, to which 0.1% sucrose has been added, into 16×75 mm test tubes. Sucrose is added to saliva on the first day only. Place the rod holders in a modified 37° C. incubator designed to dip roughened glass rods into test tubes to a depth of 1.5 cm at 1 rpm. Dip rods overnight. The design of the incubator is fully shown in Attachment 1. Prepare plaque growth media described above and autoclave for Day 2 (saliva is added on Day 2 before use).

### 4. Day 2:

In the morning, place rods in 7 ml of plaque growth media. Dip rods for six hours and then place back into saliva for overnight dipping. Prepare glycolysis buffer media as described above and autoclave for Day 3.

### 5. Day 3:

On the morning of the third day, prepare dentifrice slurries (1 part dentifrice to 3 parts water) and place in 16×75 mm test tubes in the first row of the treatment rack adding enough slurry to fully cover plaque growth on rods (approximately 7 ml). In the second and third rows, place 17×100 mm test tubes containing 10 ml of DI water for washing the rods after treatment. Remove the rods from the incubator and treat for 60 secs, dipping once per second in the dentifrice slurries using the treatment rack. Wash the rods with 2×10 secs dippings in the test tubes containing the DI water. Place the rods back in the incubator and dip in 16×75 mm test tubes containing 7 ml of glycolysis media. In the afternoon, approximately 6 hours following treatment, measure and record the pH of the reference glycolysis buffer and the pH of the glycolysis buffer containing the treated plaque rods.

### 9. Controls:

Usual controls for a dentifrice i-PGRM include Crest Cavity Protection (negative control) and Crest Gum Care (positive control). For experiments on mouth rinses or solutions, usual controls include water, placebo mouth rinse formulations (negative controls) and Peridex mouthwash (positive control). There are 4 rods per treatment group with a maximum capacity of 12 treatments that includes the controls.

### 10. Results:

Average the four pH values for each treatment group and report as a mean pH value. Final results are reported as percent efficacy relative to positive (100%) and negative control (0%) according to the following equation,

% Efficacy versus positive control=(1−(Avg pH positive control−Avg pH Test)/(Avg pH positive control−Avg pH negative control))*100

with efficacy demonstrated at 50%, preferably 60%, more preferably 70%, and most preferably 80% and higher.

### Powder Stain Prevention Model (PSPM)

The Powder Stain Prevention Model (PSPM) is a screening technique where hydroxyapatite powder (HAP) is used as a substrate for stain accumulation. The general purpose of this technique is to illustrate and quantify the stain prevention ability or staining potential of chemical agents used in oral care. Hydroxyapatite powder provides a large surface area to which tea chromogens adsorb. Pretreatment of HAP with oral care actives, either in rinse or dentifrice form, results in different levels of stain accumulation depending upon the ability of the actives to block or enhance the binding of these chromogens onto HAP surface. The magnitude of stain can then be quantified by image analysis. Steps involved in PSPM are described below.

### 1. HAP Pretreatment

Measure 200 mg-210 mg of HAP powder (Bio-Gel® HTP-Gel Catalog #130-0421, Bio-Rad Laboratories (Hercules, Calif.) into 50 ml centrifuge tubes. Add 20 ml of treatment (supernatant of 1:3 paste to water slurry) to each tube. Tube is vortexed for 30 seconds to fully suspend HAP in treatment followed by centrifugation at 15,000 rpm for 15 mins. After centrifugation, supernatant is decanted and pellet redistributed by adding 25 ml of water, vortexing, centrifuging at 15,000 rpm for 15 mins, and decanting—making sure pellet breaks up during vortexing. The wash cycle is repeated two more times.

### 2. HAP Staining

After final water wash, 20 ml of filtered tea (1 Lipton tea bag per 100 ml of hot water seeped for 5 minutes, filtered and used at 50° C.) is added to each pellet and vortexed for 30 seconds to fully suspend HAP in tea. Powder suspension is centrifuged at 15,000 rpm for 15 mins and decanted. About 25 ml of water is added to the tube, vortexed and then centrifuging at 15,000 rpm for 15 mins. The liquid is decanted and wash cycle is repeated 2 more times.

### 3. HAP Prep for Color Analysis

Vortex pellet in approximately 10 ml of water until fully suspended followed by filtering under vacuum onto a Millipore filter disk (Membrane Filters 4.5 μm, 47 mm Catalog # HAWP04700, Millipore Corporation, Bedford, Mass.). Prepare a control disk using˜200 mg of untreated, unstained HAP. Filter disks are then dried overnight in flat position and then laminated.

### 4. Color Analysis of Stained HAP

Calibrate Spectra Scan PR650 chromameter (Kyokko Trading Co., Ltd., Japan) according to instructions. Read untreated HAP control and HAP treatments for color measuring L*a*b* (CIELAB color space) using HyperTerminal software package (version 595160 (Hilgraeve, Inc.)).

### 5. Controls

Usual controls for a dentifrice PSPM are water, and Crest Cavity Protection (negative control). For the evaluation of stannous fluoride containing formulations either Crest Gum Care or Colgate GelKam are used as positive controls.

### 6. Results:

Calculate changes in L* (brightness), a* (red(+)/green(−)), b* (yellow (−)/blue(+)), and in E (total color) as follows:

ΔL=L*_{treated HAP}−L*_{untreated HAP}

Δa=a*_{treated HAP}−a*_{untreated HAP}

Δb=b*_{treated HAP}−b*_{untreated HAP}

ΔE=square root((ΔL)²+(Δa)²+(Δb)²)

Report results as average ΔL, Δa, Δb, and/or ΔE and percent prevention of stain (ΔL & ΔE) versus the negative control.

### F HAP Uptake Model

The F Uptake model is an in vitro screening model. Hydroxyapatite powder (HAP) is used in this model as a substrate. HAP is treated with a solution or dentifrice supernatant. Then HAP is dissolved in acid solution and the soluble fluoride (F) measured using an F electrode to quantify the amount of soluble F uptake on the surface of the HAP powder. It is generally considered a quick way to test how the ingredients in a given composition impact the anti cavity performance. Steps involved in FHAP uptake model are described below.

### 1. HAP Pretreatment

Measure 600 mg of HAP powder (Bio-Gel® HTP-Gel Catalog #130-0421, Bio-Rad Laboratories (Hercules, Calif.) into 50 ml centrifuge tubes. Add 20 ml of treatment (supernatant of 1:9 paste to water slurry) to each tube. Tube is vortexed for 2 minutes to fully suspend HAP in treatment followed by centrifugation at 15,000 rpm for 15 minutes. After centrifugation, supernatant is decanted and pellet redistributed by adding 25 ml of water, vortexing, centrifuging at 15,000 rpm for 15 minutes, and decanting—making sure pellet breaks up during vortexing. The wash cycle is repeated one more times. After final water wash, decant the supernatant and allow the pre-treated HAP powder to dry overnight in an open oven at maintained at 37° C.

### 2. HAP Sample Preparation for Fluoride Measurement

Weigh 50 mg of pre-treated HAP powder in 15 ml centrifuge tubes and then add 10 ml of 1N hydrochloric acid into each tube and vortex to dissolve the HAP powder.

### 3. Fluoride Analysis

After complete dissolution of HAP, mix 1 ml of the HAP solution with 0.75 ml of 1N NaOH solution in a 15 ml centrifuge tube. To this add 1.75 ml 0.2N EDTA/0.2N THAM buffer and measure fluoride via fluoride ion specific electrode. Fluoride reference solutions are use to generate calibration curve which was then used to calculate concentration of fluoride in HAP pre-dissolved solution. After taking into account the dilution factor concentration of fluoride is divided by weight of HAP powder used (600 mg) to calculate amount of fluoride adsorbed per gram of HAP powder, and reported as the final result.

### Comparative Compositions

The commercially available (at one point in time) dentifrice compositions Crest Cavity Protection (“Whitebox”), Crest Pro-Health, and Crest Gum Care set forth below were used for comparison and/or positive and/or negative controls in the tests conducted herein. The formulations used in the tests herein are set forth below and are made by standard making procedures.

X1: Crest Cavity Protection - Crest Regular
Toothpaste/“Whitebox”

| Ingredient | % |
|---|---|
| Sorbitol USP (70% sol'n) | 65.508 |
| Water | QS |
| Silica (USP) | 15.000 |
| Flavor 55830 | 0.810 |
| Trisodium phosphate decahydrate | 1.100 |
| Sodium lauryl sulfate (28% sol'n) | 4.000 |
| Monosodium phosphate monohydrate | 0.419 |
| Titanium dioxide (Rutile USP) | 0.525 |
| CMC Sodium USP | 0.750 |
| Carbomer 956 | 0.300 |
| Sodium fluoride (USP) | 0.243 |
| Sodium saccharin (USP) | 0.130 |
| FD&C Blue #1 (Sol'n) | 0.050 |
| TOTAL | 100.000 |

X2: Crest Pro-Health (Clean Mint)

| Ingredient | Wt % |
|---|---|
| Stannous Fluoride, USP | 0.454 |
| Glycerin USP 99.7% Veg Based-Syn | 36.944 |
| PROPYLENE GLYCOL USP | 7.000 |
| CREST | |
| POLYETHYLENE GLYCOL 300, | 7.000 |
| NF (478 | |
| Water, USP | QS |
| SILICA, DENTAL-TYPE NF Z-119 | 12.500 |
| Silica, Z109, NF | 12.500 |
| Sodium Polyphosphate, FCC (Glass | 13.000 |
| H) | |
| XANTHAN GUM (KELTROL 1000) | 0.250 |
| Carrageenan Mixture (Iota) | 0.600 |
| SODIUM LAURYL SULFATE | 3.400 |
| (48397-002 | |
| FLAVOR, 49101 TEABERRY | 1.000 |
| SACCHARIN SODIUM USP | 0.500 |
| GRANULAR, HI | |
| Dye, FD&C Blue #1 Soln (1.0%) | 0.300 |
| Microwhite, Polyethylene Specks | 0.300 |
| SODIUM PHOSPHATE, TRIBASIC | 1.100 |
| DODEC | |
| ZINC Lactate Dihydrate | 2.500 |
| Sodium Gluconate, USP | 0.652 |

*Precipitated silicas Z109 and Z1129 are commercially available from Huber, USA.

X3: Crest Gum Care

| Ingredient | Wt % |
|---|---|
| Stannous Fluoride, USP | 0.454 |
| Sorbitol Solution, USP (70% LRS) | 55.159 |
| Silica Abrasive USP (Z119) | 20 |
| Purified Water, USP | 13 |
| Sodium Lauryl Sulfate (28% Sol'n) | 4 |
| Sodium Gluconate, USP | 2.082 |
| Stannous Chloride Dihydrate, FCC | 1.5 |
| Flavor 55995 | 1 |
| Carrageenan Mixture | 0.8 |
| Sodium Hydroxide solution, FCC (50%) | 0.8 |
| Hydroxyethylcellulose, NF (Medium Visc.) | 0.5 |
| Saccharin Sodium, USP | 0.455 |
| FD&C Blue #1 Color solution, Non-alcohol | 0.25 |
| TOTAL | 100 |

### Example I

### Dentifrice Compositions

Example I illustrates compositions in Table 1a containing stannous in combination with GANTREZ and zinc in combination with stannous and GANTREZ and shows the effects of excess stannous deposition and it's modulation by GANTREZ on permeation and uptake of fluoride.

These compositions may be suitably prepared by conventional methods chosen by the formulator and have been found to provide superior fluoridation, protection against enamel demineralization, antibacterial activity and reduced staining.

TABLE 1a

| | 1A | 1B | 1C | 1D | 1E | 1F |
|---|---|---|---|---|---|---|
| | Formula | | | | | |
| | 1100 | 1100 | 2800 | 2800 | 1100 NaF/ | 1100 SnF2/ |
| | NaF | SnF2 | NaF | SnF2 | 2% GNTZ | 2% GNTZ |
| Stannous Fluoride | — | 0.45 | — | 1.15 | — | 0.45 |
| Sodium Fluoride | 0.24 | | 0.62 | | 0.24 | — |
| Sorbitol (70% Soln) | 42.1 | 42.1 | 42.1 | 42.1 | 42.1 | 42.1 |
| GANTREZ S-95 (35% soln.) | — | — | — | — | 5.71 | 5.71 |
| Zinc Lactate | — | — | — | — | — | — |
| Sodium Gluconate | 1.06 | 1.06 | 1.06 | 1.06 | 1.06 | 1.06 |
| Sodium Saccharin | 0.80 | 0.80 | 0.80 | 0.80 | 0.80 | 0.80 |
| Silica Z109 | 15 | 15 | 15 | 15 | 15 | 15 |
| Hydroxyethylcellulose | 1.00 | 1.00 | 1.00 | 1.00 | 1.00 | 1.00 |
| Carrageenan | 0.70 | 0.70 | 0.70 | 0.70 | 0.70 | 0.70 |
| Carboxymethyl cellulose | 1.30 | 1.30 | 1.30 | 1.30 | 1.30 | 1.30 |
| SLSS (27.9% soln) | 5.00 | 5.00 | 5.00 | 5.00 | 5.00 | 5.00 |
| Sodium Hydroxide (50% | 1.4 | 1.50 | 1.4 | 1.50 | 1.4 | 1.4 |
| soln) | | | | | | |
| Flavor | 1.2 | 1.00 | 1.2 | 1.00 | 1.2 | 1.2 |
| Dye | 0.20 | 0.20 | 0.20 | 0.20 | 0.20 | 0.20 |
| USP Water | 25.9 | 25.9 | 25.9 | 25.9 | 25.9 | 25.9 |
| Total | 100.0 | 100.0 | 100.0 | 100.0 | 100.0 | 100.0 |
| Metal/GNTZ (wt ratio) | | | | | | 0.17 |

### Enamel Fluoridation Data

Stannous effects on fluoridation—excess surface deposition of stannous leads to reduction to fluoride uptake even though fluoride level is more than doubled. An in-vitro pH cycling study was conducted to evaluate the effect of excess metals and its modulation on fluoride uptake in pre-demineralized human enamel specimens. (Details of method is provided in U.S. Pat. No. 7,387,774)

TABLE 1b

| | Treatment | μg F/cm\hat{ }2 |
|---|---|---|
| 1A | 1100 NaF | 24.082 |
| 1B | 1100 SnF2 | 20.294 |
| 1C | 2800 NaF | 56.808 |
| 1D | 2800 SnF2 | 18.164 |

Stannous complexation with GANTREZ leads to controlled deposition of stannous on enamel surface resulting in higher fluoride uptake at same fluoride levels—GANTREZ does not impact fluoride uptake from a NaF containing formula.

TABLE 1c

| | Treatment | μg F/cm\hat{ }2 |
|---|---|---|
| 1A | 1100NaF | 24.082 |
| 1E | 1100 NaF/2% GANTREZ | 22.510 |
| 1B | 1100 SnF2 | 20.294 |
| 1F | 1100 SnF2/2% GANTREZ | 31.202 |

### Example 2

### Dentifrice Compositions

The compositions in Table 2a and the corresponding data highlight the effects of excess zinc deposition and it's modulation by GANTREZ on permeation and uptake of fluoride. These compositions may be suitably prepared by conventional methods chosen by the formulator and have been found to provide superior fluoridation, protection against enamel demineralization, antibacterial activity and reduced staining

TABLE 2a

| | Formula | | | |
|---|---|---|---|---|
| | | | | 2D |
| | | 2B | | 1100 |
| | | 1100 NaF/ | 2C | SnF2/ |
| | 2A | 1% | 1100 | 1% |
| | 1100 NaF/ | ZnL/2% | SnF2/ | ZnL/2% |
| | 1% ZnL | GNTZ | 1% ZnL | GNTZ |
| Stannous Fluoride | — | — | 0.45 | 0.45 |
| Sodium Fluoride | 0.24 | 0.24 | — | — |
| Sorbitol (70% Soln) | 42.1 | 42.1 | 42.1 | 42.1 |
| GANTREZ S-95 (35% soln.) | — | 5.71 | — | 5.71 |
| Zinc Lactate | 1.00 | 1.00 | 1.00 | 1.00 |
| Sodium Gluconate | 1.06 | 1.06 | 1.06 | 1.06 |
| Sodium Saccharin | 0.80 | 0.80 | 0.80 | 0.80 |
| Silica Z109 | 15 | 15 | 15 | 15 |
| Hydroxyethylcellulose | 1.00 | 1.00 | 1.00 | 1.00 |
| Carrageenan | 0.70 | 0.70 | 0.70 | 0.70 |
| Carboxymethyl cellulose | 1.30 | 1.30 | 1.30 | 1.30 |
| SLSS (27.9% soln) | 5.00 | 5.00 | 5.00 | 5.00 |
| Sodium Hydroxide (50% | 1.4 | 1.50 | 1.50 | 1.4 |
| soln) | | | | |
| Flavor | 1.2 | 1.00 | 1.00 | 1.2 |
| Dye | 0.20 | 0.20 | 0.20 | 0.20 |
| USP Water | 25.9 | 25.9 | 25.9 | 25.9 |
| Total | 100.0 | 100.0 | 100.0 | 100.0 |
| Zn/GNTZ (wt ratio) | | 0.5 | | 0.12 |
| Sn/GNTZ (wt ratio) | | — | | 0.17 |
| Sn + Zn/GNTZ (wt ratio) | | — | | 0.29 |

### Enamel Fluoridation Study

GANTREZ complexation with zinc minimizes the negative effects of excess zinc on mineralization and fluoride uptake and leads to higher fluoride uptake at same fluoride levels.

TABLE 2b

| | Treatment | μg F/cm\hat{ }2 |
|---|---|---|
| 2A | 1100NaF/1% ZnL | 18.958 |
| 2B | 1100NaF/1% ZnL/2% GANTREZ | 42.284 |
| 2C | 1100SnF2/1% Zn | 14.511 |
| 2D | 1100SnF2/1% ZnL/2% GANTREZ | 41.604 |

### Example 3

### Dentifrice Compositions

The compositions in Table 3a and the corresponding data highlight surface protection benefit of stannous is maintained while excess surface deposition is controlled by GANTREZ to enhance fluoride uptake and mitigate stain. These compositions may be suitably prepared by conventional methods chosen by the formulator and have been found to provide superior fluoridation, protection against enamel demineralization, antibacterial activity and reduced staining.

TABLE 3a

| | Formula | | | |
|---|---|---|---|---|
| | | | 3C | |
| | | | 1100 | |
| | | | SnF2/ | 3D |
| | | 3B | 1% | 1100 SnF2/ |
| | 3A | 2800 | ZnL/2% | 0.25% ZnL/ |
| | 1100 NaF | NaF | GNTZ | 2% GNTZ |
| Stannous Fluoride | — | — | 0.45 | 0.45 |
| Sodium Fluoride | 0.24 | 0.62 | — | — |
| Sorbitol (70% Soln) | 42.1 | 42.1 | 42.1 | 42.1 |
| GANTREZ S-95 (35% soln.) | — | — | 5.71 | 5.71 |
| Zinc Lactate | — | — | 1.00 | 0.25 |
| Sodium Gluconate | 1.06 | 1.06 | 1.06 | 1.06 |
| Sodium Saccharin | 0.80 | 0.80 | 0.80 | 0.80 |
| Silica | 15 | 15 | 15 | 15 |
| Hydroxyethylcellulose | 1.00 | 1.00 | 1.00 | 1.00 |
| Carrageenan | 0.70 | 0.70 | 0.70 | 0.70 |
| Carboxymethyl cellulose | 1.30 | 1.30 | 1.30 | 1.30 |
| SLSS (27.9% soln) | 5.00 | 5.00 | 5.00 | 5.00 |
| Sodium Hydroxide (50% | 1.4 | 1.4 | 1.4 | 1.4 |
| soln) | | | | |
| Flavor | 1.2 | 1.2 | 1.2 | 1.2 |
| Dye | 0.20 | 0.20 | 0.20 | 0.20 |
| USP Water | 25.9 | 25.9 | 25.9 | 25.9 |
| Total | 100.0 | 100.0 | 100.0 | 100.0 |
| Zn/GNTZ (wt ratio) | | | 0.12 | 0.03 |
| Sn/GNTZ (wt ratio) | | | 0.17 | 0.17 |
| Sn + Zn/GNTZ (wt ratio) | | | 0.29 | 0.20 |

### Enamel Solubility Reduction Study

GANTREZ complexation with zinc minimizes the negative effects of excess zinc on mineralization and fluoride uptake and leads to higher fluoride uptake at same fluoride levels.

TABLE 3b

| | Treatment | ESR (% Red) |
|---|---|---|
| 3A | 1100 NaF | 10.6 |
| 3B | 2800 ppm | 13.3 |
| 3C | Crest Pro-Health | 25.3 |
| 3D | 1100 SnF2/1% ZnL/GANTREZ | 45 |
| 3E | 1100 | 42 |
| | SnF2/0.25% ZnL/GANTREZ | |

### Example 4

### Dentifrice Compositions

Dentifrice compositions according to the present invention are set forth in Tables 4a and 4b below. These compositions may be suitably prepared by conventional methods chosen by the formulator and have been found to provide superior fluoridation, protection against enamel demineralization, antibacterial activity and reduced staining.

TABLE 4a

| | 4A | |
|---|---|---|
| | % (by | 4B |
| INGREDIENTS | weight) | % (by weight) |
| Stannous Fluoride | 0.4540 | |
| USP Water | 25.2000 | 25.2000 |
| Sorbitol (70%) | 37.6320 | 37.6320 |
| GANTREZ S-95 (35% soln.) | 5.7100 | 5.7100 |
| Zinc Lactate | 1.0000 | 1.0000 |
| Sodium Gluconate | 1.0640 | 1.0640 |
| Sodium Saccharin | 0.8000 | 0.8000 |
| Z109 Amorphous Silica gel | 15.0000 | 15.0000 |
| Hydroxyethylcellulose 250M 420, NF | 0.7200 | 0.7200 |
| Carrageenan | 1.0800 | 1.0800 |
| Xanthan Gum, NF | 0.5400 | 0.5400 |
| Xylitol | 3.0000 | 3.0000 |
| SLSS (27.9%) | 5.0000 | 5.0000 |
| Sodium Hydroxide (50%) | 1.4000 | 1.4000 |
| Wintergreen Flavor | 1.2000 | 1.2000 |
| Dye Sol′n FD&C Blue #1 | 0.2000 | 0.2000 |
| TOTAL | 100.0000 | 100.0000 |

GANTREZ S-95 - a polycommercially available from Ashland Chemicals
Z109 - A precipitated silica abrasive available from Huber

TABLE 4b

| | 4C |
|---|---|
| INGREDIENT | % (by weight) |
| Stannous Fluoride | 0.454 |
| USP Water | 25.500 |
| Sorbitol (70%) | 37.452 |
| GANTREZ S-95 | 5.710 |
| Zinc Lactate | 0.250 |
| Sodium Gluconate | 1.064 |
| Sodium Saccharin | 0.800 |
| Z109 Amorphous Silica gel | 15.000 |
| Hydroxyethylcellulose | 0.720 |
| Carrageenan | 1.080 |
| Xanthan Gum | 0.540 |
| Xylitol | 3.000 |
| SLSS (27.9%) | 5.000 |
| Sodium Hydroxide (50%) | 1.200 |
| HOPS CLEAN BETA BIO (45%) | 0.330 |
| Titanium Dioxide | 0.500 |
| Titan Peppermint | 1.200 |
| Dye Sol′n FD&C Blue #1 | 0.200 |
| Total | 100.000 |
| TOTAL | |

HOPS CLEAN BETA BIO - commercially available from Hopsteiner

TABLE 4c
Molar %

| | SnF2 | Sn | | ZnL | | Zn | GANTREZ | GANTREZ |
|---|---|---|---|---|---|---|---|---|
| | (%) | (%) | Sn (M) | (%) | Zn (%) | (M) | (%) | (M) |
| 4A | 0.4540 | 0.3400 | 0.0038 | 1.0000 | 0.2400 | 0.0037 | 2 | 0.000 |
| 4B | 0.4540 | 0.3400 | 0.0038 | 0.2500 | 0.0600 | 0.0009 | 2 | 0.000 |

TABLE 4d
Molar Ratios

| | | Sn/Zn | Sn/GNTZ |
|---|---|---|---|
| | Sn + Zn (M) | (M ratio) | (M ratio) |
| 4A | 0.0075 | 1.04 | 413.60 |
| 4B | 0.0048 | 4.17 | 413.60 |

TABLE 4e
Weight %

| | SnF2 | | | | Sn + Zn | GANTREZ |
|---|---|---|---|---|---|---|
| | (%) | Sn (%) | ZnL (%) | Zn (%) | (%) | (%) |
| 4A | 0.4540 | 0.34 | 1.0 | 0.24 | 0.58 | 2 |
| 4B | 0.4540 | 0.34 | 0.25 | 0.06 | 0.40 | 2 |

TABLE 4f
Weight % Ratios

| | Sn/Zn | Sn/GNTZ | Zn/GNTZ (wt | (Sn + Zn)/GNTZ |
|---|---|---|---|---|
| | (wt % ratio) | (wt % ratio) | % ratio) | (wt % ratio) |
| 4A | 1.42 | 0.170 | 0.169 | 0.290 |
| 4B | 5.67 | 0.170 | 0.011 | 0.200 |

### Example 5

### COMPARATIVE EXAMPLES

### Dentifrice

Comparative examples 5A-5F are shown in Table 5 and may be suitably prepared by conventional methods chosen by the formulator

TABLE 5

| Ingredient | 5A | 5B | 5C | 5D | 5E | 5F |
|---|---|---|---|---|---|---|
| Stannous Fluoride | 0.454 | 0.454 | 0.454 | 0.45 | 0.45 | — |
| Water | — | — | — | — | 30 | 30 |
| Stannous Chloride | | | | | | 1.5 |
| Sodium | 13 | — | 13 | 7 | — | — |
| Polyphosphate | | | | | | |
| Sodium phosphate tribasic | 1.1 | 1.1 | 1.1 | 1.1 | 1.1 | 1.1 |
| Sorbitol 70% soln. | — | — | — | — | 20 | 20 |
| GANTREZ (5.71 * | — | 2 | — | 2 | 2 | 2 |
| 0.35 ??) | | | | | | |
| Zinc Lactate | 2.5 | 1.5 | — | 2 | — | 2 |
| Sodium Gluconate | 0.652 | 0.652 | 0.652 | 0.652 | 0.652 | 0.652 |
| Zinc carbonate | — | — | — | — | 2 | — |
| Calcium chloride | — | — | 0.423 | — | — | |
| Sodium Saccharin | 0.5 | 0.5 | 0.5 | 0.5 | 0.5 | 0.5 |
| Sodium fluoride | — | — | — | — | — | 0.24 |
| Silica | 20 | 20 | 20 | 20 | 20 | 20 |
| Propylene Glycol | 7 | 7 | 7 | 7 | — | — |
| Polyethylene Glycol | 7 | 7 | 7 | 7 | — | — |
| Glycerin | 42.244 | 54.244 | 44.321 | 46.74 | 16.89 | 15.61 |
| Poloxamer 407 | — | — | — | — | — | — |
| Hydroxyethylcellulose | | | | | | |
| Carboxymethyl | — | — | — | — | 0.25 | 0.25 |
| cellulose | | | | | | |
| Carrageenan | 0.6 | 0.6 | 0.6 | 0.6 | — | — |
| Xanthan Gum | 0.25 | 0.25 | 0.25 | 0.25 | 0.25 | 0.25 |
| Sodium Lauryl Sulfate | 3.4 | 3.4 | 3.4 | 3.4 | 4.3 | 4.3 |
| 27.9% soln. | | | | | | |
| Xylitol | | | | | | |
| Sodium Hydroxide | | | | | | |
| (50%) | | | | | | |
| Flavor | 1 | 1 | 1 | 1 | 1.3 | 1.3 |
| FD&C Blue # 1 1% | 0.3 | 0.3 | 0.3 | 0.3 | 0.3 | 0.3 |
| soln. | | | | | | |
| Titanium Dioxide | | | | | | |
| HOPS Clean Beta Bio | | | | | | |
| Total | 100 | 100 | 100 | 100 | 100 | 100 |
| Sn/GNTZ ratio | | 0.17 | | | 0.17 | 0.39 |
| Zn/GNTZ ratio | | 0.18 | | | 0.52 | 0.24 |
| Sn + Zn/GNTZ ratio | | 0.35 | | | 0.69 | 0.63 |

### Example 6

### Dentifrice Compositions

Dentifrice oral care compositions according to the present invention incorporating itaconic acid polymers are provided in Table 6. Dentifrice formulations C through E were prepared by traditional methods, differing only by the type of polymer used. The making procedure of the dentifrices herein is as follows: Add half of sorbitol, color solution, and half of flavor to dentifrice making mixing tank. Close the lid and pull vacuum to 19+/−1 7 mm Hg and start agitator at 32+/−2 rpm. Temperature of vessel is at 95° F. Add sodium gluconate, stannous fluoride and Hydroxyethyl cellulose to the vessel by cycling vacuum to incorporate powders and homogenize. Following powder addition, slowly add water and rest of Sorbitol solution. Mix and deaerate until hydroxyethyl cellulose is fully hydrated. Add polymer solution. When completely mixed, add NaOH solution and continue mixing to ensure complete addition to the batch. Weigh and add the rest of the solids (e.g., saccharin sodium, zinc lactate, Xanthan gum, Carrageenan, sucralose and one third of silica) by cycling vacuum to the mixing vessel and homogenize. Increase the vacuum to deaerate contents of the vessel and finally add the rest of silica. Once silica addition is complete, add sodium lauryl sulfate solution and flavor with continued mixing and dearation. Complete a final homogenization cycle to ensure product homogeneity and then pump out the product.

TABLE 6
Dentifrice Compositions Containing Itaconic Acid Polymers

| | | | 2% |
|---|---|---|---|
| | 2% | 2% | ITACONIX |
| | ITACONIX | ITACONIX | DSP2K PPT |
| | DSP1K | DSP2K | Silica |
| INGREDIENTS | Formula C | Formula D | Formula E |
| Sorbitol (70%) | 33 | 33 | 33 |
| Sodium Saccharin | 0.80 | 0.80 | 0.80 |
| Teco Fused Silica | 15.0 | 15.0 | — |
| Silica Z109 | — | — | 15 |
| Hydroxyethyl- | 1.0 | 1.0 | 1.0 |
| cellulose | | | |
| Carrageenan | 0.70 | 0.70 | 0.70 |
| CMC 7M8SF P&G | 1.30 | 1.30 | 1.30 |
| SLSS (27.9%) | 5.0 | 5.0 | 5.0 |
| Dye Sol′n FD&C | 0.2 | 0.2 | 0.2 |
| Blue #1 | | | |
| Zn Lactate | 1 | 1 | 1 |
| SnF2 | 0.45 | 0.45 | 0.45 |
| Na Gluconate | 1.06 | 1.06 | 1.06 |
| DSP1K 48% active | 4.17 | — | — |
| soln | | | |
| DSP2K 48% active | — | 4.17 | 4.17 |
| soln | | | |
| Wintergreen flavor | 1.2 | 1.2 | 1.2 |
| NaOH 50% soln | — | — | — |
| HCL (37%) | 0 | 0 | 0 |
| USP Water | QS | QS | QS |
| Total | 100 | 100 | 100 |

1. Itaconic polymers useful herein are the homopolymers of itaconic acid (methylenesuccinic acid), having a molecular weight of from about 1000 to about 20,000. Such polymers are commercially available from ITACONIX, USA, for example, the linear polyitaconic acid partially neutralized with sodium salt polymers sold under the name ITACONIX DSP2K (molecular weight about 5,000; CAS #26099-89-8), ITACONIX DSP1K (molecular weight about 2,000), ITACONIX DSP5K, and ITACONIX DSP10K.

### Stain Prevention Test

These formulations above, along with positive control CREST GUM CARE (a high-stannous dentifrice formulation known to cause tooth staining in some consumers) and commercially available negative control Crest Cavity Protection (a sodium fluoride and precipitated silica dentifrice) were tested using the iPSTM (pellicle substrate) and PSPM (HAP powder substrate) methods and the results tabulated in Table 7 and Table 8 below. Table 7: iPTSM study of ITACONIX Polymer comparison with CREST GUM CARE and CREST CAVITY PROTECTION in a dose response manner for stain control benefits in dentifrice formulations.

| | | Rel. Staining | Stat. |
|---|---|---|---|
| Treatment | Polymer | Potential [%] | grouping |
| Crest Cavity Protection - | — | 0 | C |
| negative control | | | |
| CREST GUM CARE - positive | — | 100 | A |
| control | | | |
| Formula C | 2% DSP1K | −10 | C |
| Formula D | 2% DSP2K | −4 | C |
| Formula E??? | 2% DSP2K | | |

As may be seen, Polymer DSP2K supplied by ITACONIX showed improved stain control benefit versus CREST GUM CARE in the iPTSM model.

### Efficacy Performance Test

More than often, metal complexing agents and chelants, while provide good stain control benefits, can significantly reduce bioavailibility of metals and the efficacy associated with them at given doses. Hence, it is important to determine if there is any reduction in performance of stannous in the presence of these new polymers. Dentifrice formulations provided in Table 1 were tested for enamel fluoridation and antibacterial activities to determine if anticaries and antibacterial performance of the formulations is maintained in the presence of new copolymers relative to GANTREZ S-95.

An iPGRM model was employed for antibacterial performance assessment of the dentifrice compositions containing itaconic acid homopolymers versus CREST CAVITY PROTECTION and CREST GUM CARE. The results of the iPGRM model results are shown in Table 8.

TABLE 8
iPGRM Model Compiled Results (Percent anti-glycolytic
efficacy: Gum Care = 100%, Crest Cavity Protection = 0%)

| Formula Treatment | Polymer | % Efficacy |
|---|---|---|
| Formula C | 2% DSP1K F—Si | |
| Formula D | 2% DSP2K F—Si | |
| Formula E | 2% DSP2K PPT | |
| | Silica | |
| Crest cavity Protection | — | 0 |
| Crest Gum care | — | 100 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition having improved efficacy for preventing and/or reducing caries, said composition comprising:
a) at least about 0.005%, by weight of the composition, of metal ions wherein the composition comprises at least about 0.005%, by weight of the composition, of zinc ions and optionally comprises from about 0.001% to about 3%, by weight of the composition, of stannous ions;
b) at least about 100 ppm, by weight of the composition, of fluoride ions;
c) from about 0.03% to about 10%, by weight of the composition, of a mineral surface active agent selected from homopolymers of itaconic acid, homopolymers of acrylic acid, copolymers of maleic acid and acrylic acid, copolymers of maleic anhydride or acid with methyl vinyl ether, and mixtures thereof;
d) at least 5%, by weight of the composition, of water;
e) less than 10%, by weight of the composition, of fused silica, calcium based abrasives, and mixtures thereof;
f) less than 5%, by weight of the composition, of polyphosphates having n+3 or higher; and
wherein the weight ratio of metal ion to the mineral surface active agent is equal to or less than about 0.5.

2. An oral care composition according to claim 1, wherein the composition comprises from about from about 0.01% to about 2%, by weight of the composition, of the metal ions wherein the composition comprises from about 0.005% to about 1%, by weight of the composition, of zinc ions and optionally comprises from about 0.01% to about 1.5%, by weight of the composition, of stannous ions.

3. An oral care composition according to claim 1, wherein the composition comprises from about 100 ppm to about 15,000 ppm, by weight of the composition, of the fluoride ions.

4. An oral care composition according to claim 1, wherein the weight ratio of the metal ion to the mineral surface active agent is equal to or less than about 0.3.

5. An oral care composition according to claim 1, wherein the composition comprises from about 0.03% to about 10%, by weight of the composition, of the mineral surface active agent.

6. An oral care composition according to claim 1, wherein the mineral surface active agent is selected from homopolymers of itaconic acid.

7. An oral care composition according to claim 1, wherein the mineral surface active agent is selected from copolymers of maleic anhydride or acid with methyl vinyl ether.

8. An oral care composition according to claim 1, wherein the mineral surface active agent is selected from copolymers of maleic acid and acrylic acid.

9. An oral care composition according to claim 1, wherein the composition comprises less than 1%, by weight of the composition, of fused silica, calcium based abrasives, and mixtures thereof.

10. An oral care composition according to claim 1, wherein the composition comprises less than 1%, by weight of the composition, of polyphosphates.

11. An oral care composition according to claim 1, wherein the composition further comprises at least one botanical or extract thereof selected from chamomile, cinnamon, citrus, clove, echninacea, eucalyptus, fennel, ginger, green tea, hop, magnolia, nutmeg, peppermint, pomegranate, rosemary, saffron, sage, spearmint, star anise, turmeric, wintergreen, extracts thereof and mixtures thereof.

12. An oral care composition according to claim 11, wherein the composition the botanical or extract thereof is selected from Hops, extracts thereof and mixtures thereof.

13. An oral care composition according to claim 1, wherein the composition further comprises a stannous-compatible abrasive.

14. An oral care composition according to claim 13, wherein the abrasive is selected from precipitated silica, polymethylsilsesquioxane silicone resin particles, and mixtures thereof.

15. An oral care composition according to claim 1, wherein the composition comprises a zinc ion source selected from zinc citrate, zinc sulfate, zinc glycinate, sodium zinc citrate, zinc lactate, and mixtures thereof.

16. An oral care composition according to claim 15 wherein the zinc ion source is selected from zinc citrate, zinc lactate, and mixtures thereof.

17. An oral care composition according to claim 16, wherein the zinc ion source is zinc lactate.

18. An oral care composition according to claim 1, wherein the composition comprises a stannous ion source selected from stannous fluoride, stannous chloride, stannous pyrophosphate, stannous chloride dihydrate, stannous acetate, stannous gluconate, stannous oxalate, stannous sulfate, stannous lactate, stannous tartrate, and mixtures thereof.

19. An oral care composition according to claim 18, wherein the stannous ion source is selected from stannous fluoride, stannous chloride, and mixtures thereof.

20. An oral care composition according to claim 18, wherein the stannous ion source is stannous fluoride and the zinc ion source is zinc lactate.

21. An oral care composition having improved efficacy for preventing and/or reducing caries, said composition comprising:
a) from about from about 0.05% to about 1%, by weight of the composition, of metal ions wherein the composition comprises from about 0.05% to about 1%, by weight of the composition, of zinc ions and optionally comprises from about 0.01% to about 1.5%, by weight of the composition, of stannous ions.
b) from about 100 ppm to about 15,000 ppm, by weight of the composition, of fluoride ions;
c) from about 0.03% to about 10%, by weight of the composition, of a mineral surface active agent selected from homopolymers of itaconic acid, homopolymers of acrylic acid, copolymers of maleic acid and acrylic acid, copolymers of maleic anhydride or acid with methyl vinyl ether, and mixtures thereof;
d) at least 20%, by weight of the composition, of water;
e) less than 5%, by weight of the composition, of fused silica, calcium based abrasives, and mixtures thereof;
f) less than 5%, by weight of the composition, of polyphosphates having n+3 or higher; and
wherein the weight ratio of metal ion to the mineral surface active agent is equal to or less than about 0.5.

22. An oral care composition according to claim 1, wherein the composition further comprises at least one botanical or extract thereof selected from chamomile, cinnamon, citrus, clove, echinacea, eucalyptus, fennel, ginger, green tea, hop, magnolia, nutmeg, peppermint, pomegranate, rosemary, saffron, sage, spearmint, star anise, turmeric, wintergreen, extracts thereof and mixtures thereof.

23. An oral care composition according to claim 22, wherein the botanical or extract thereof is selected from Hops, extracts thereof and mixtures thereof.

24. A method of preventing caries, said method comprising the steps of:
a) applying the composition of any of claims 1 through 23 to the teeth on an at least daily basis for a period of at least one week;
b) rinsing the composition from the oral cavity; and
c) expectorating.

25. An oral care composition having improved efficacy for preventing and/or reducing caries, said composition comprising:
a) from about 0.01% to about 2%, by weight of the composition, of metal ions;
b) from about 100 ppm to about 15,000 ppm, by weight of the composition, of fluoride ions;
c) from about 0.03% to about 10%, by weight of the composition, of a mineral surface active agent selected from homopolymers of itaconic acid, homopolymers of acrylic acid, copolymers of maleic acid and acrylic acid; copolymers of maleic anhydride or acid with methyl vinyl ether; and mixtures thereof;
d) from about 0.005% to about 5% HOPS extract or components of HOPS extract alone or mixtures thereof.
e) at least 5%, by weight of the composition, of water;
wherein the weight ratio of total metal ion to the mineral surface active agent is equal to or less than 0.5.

26. An oral care composition according to claim 25, wherein the composition comprises at least about 0.005%, by weight of the composition, of zinc ions and optionally comprises from about 0.001% to about 3%, by weight of the composition, of stannous ions.

27. An oral care composition according to claim 25, wherein the weight ratio of total metal ion to the mineral surface active agent is equal to or less than 0.35.
